(19) United States
(12) Patent Application Publication
(10) Pub. No.: US 2008/0227864 A1
(43) Pub. Date: Sep. 18, 2008

(54) Lhr1 Activators as Medicaments

(76) Inventors: Bryan James Goodwin, Durham, NC (US); Eugene Lee Stewart, Durham, NC (US); Peter Jonathan Brown, Durham, NC (US); Philippe Delerive, Les Ulis (FR)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/598,338

(22) PCT Filed: Feb. 23, 2005
(86) PCT No.: PCT/US2005/005754

§ 371 Date: Aug. 24, 2006

Related U.S. Application Data
(60) Provisional application No. 60/547,130, filed on Feb. 24, 2004.

(51) Int. Cl. A61K 31/136; A61P 3/00; A61P 9/00

(52) U.S. Cl. 514/647

(57) ABSTRACT

Methods or prevention or treatment of diseases or conditions caused by low plasma apoA-1 levels, the use of LRH1 activators in such methods and methods for identification of compounds useful in such treatment.

### FIELD OF THE INVENTION

The present invention is concerned with medicaments. More particularly, the invention relates to compounds that bind to and activate Liver Receptor Homolog-1 (LRH1). In another aspect, the present invention relates to methods for prevention or treatment of a disease or condition caused by low plasma apoA-1 levels and methods for identifying compounds useful in the treatment of a disease or condition caused by low plasma apoa-1 levels, in particular atherosclerosis, dyslipidemia, peripheral vascular disease, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity and endotoxemia.

### BACKGROUND OF THE INVENTION

Elevated levels of circulating cholesterol are a major risk factor for the development of cardiovascular disease, the largest cause of mortality and morbidity in the industrialized world. Cholesterol homeostasis is a complex process that involves the coordinate regulation of its biosynthesis, transport, storage, and catabolism. Most mammalian cells are unable to catabolize this lipid and, thus, it is necessary for peripheral tissues to export their excess cholesterol to the liver where it is converted to bile acids or secreted into the bile as free sterol. This process is known as reverse cholesterol transport (RCT) and is particularly important in the removal of cholesterol from the vascular intima. Epidemiological, animal, and in vitro studies have demonstrated that high-density lipoprotein (HDL), which is comprised of cholesterol, cholesteryl ester, triglyceride, phospholipid, and apolipoproteins, is a central component of this pathway and levels of plasma HDL are inversely correlated to risk of cardiovascular disease (Wilson et al., 1998).

Apolipoprotein A-1 (apoA-1) is the primary structural protein component of HDL (Duverger, N., Rader, D., Duchateau, P., Fruchart, J. C., Castro, G., and Brewer, H. B., Jr (1993) Biochemistry. Vol 32, 12372-12379). A central role of this apolipoprotein in the synthesis and assembly of HDL is suggested by the tight correlation between plasma apoA-1 and plasma HDL-cholesterol levels (Rubin, E. M., Ishida, B. Y., Clift, S. M., and Krauss, R. M. (1991) Proc. Natl. Acad. Sci. USA. Vol 88, 434-438; Schaefer, E. J., Heaton, W. H., Wetzel, M. G., and Brewer, H. B., Jr (1982) Arteriosclerosis. Vol 2, 16-26). ApoA-1 is likely the physiological acceptor of free cholesterol from peripheral tissues, as lipid free apoA-1 is more efficient than plasma HDL at promoting the efflux of cholesterol from macrophage-derived foam cells (Yancey, P. G., Bielicki, J. K., Johnson, W. J., Lund-Katz, S., Palgunachari, M. N., Anantharamaiah, G. M., Segrest, J. P., Phillips, M. C., and Rothblat, G. H. (1995) Biochemistry. Vol 34, 7955-7965). Substantial support for the concept that overexpression of human apoA-1 reduces atherogenesis has been provided in studies utilising rabbits fed a high fat diet, as well as transgenic C57BL/6 and hyperlipidemic apoE knock-out mice (Duverger, N., Kruth, H., Emmanuel, F., Caillaud, J. M., Viglietta, C., Castro, G., Tailleux, A., Fievet, C., Fruchart , J. C., Houdebine, L. M., and Denefle, P. (1996) Circulation, Vol 94, 713-717; Rubin, E. M., Krauss, R. M., Spangler, E. A., Verstuyft, J. G., and Clift, S. M. (1991) Nature. Vol 353, 265-267; Plump, A. S., Scott, C. J., and Breslow, J. L. (1994) Proc. Natl. Acad. Sci. USA., Vol 91, 9607-9611; Paszty, C., Maeda, N., Verstuyft, J., and Rubin, E. M. (1994) J. Clin. Invest. Vol 94, 899-903). These studies show that increased levels of human apoA-1 in plasma effectively delay the progression of atherosclerosis. Furthermore, intravenous infusion of apoA-1/phosphatidylcholine discs rapidly increased plasma pre-b apoA-1 and HDL cholesterol concentrations in humans, through an increase in reverse cholesterol transport (Nanjee, M. N., Doran ; J. E., Lerch, P. G., and Miller N. E. (1999) Arterioscler. Thromb. Vasc. Biol. Vol 19, 979-989; Nanjee, M. N., Cooke, C. J., Garvin, R., Semeria, F., Lewis, G., Olszewski, W. L., and Miller N. E. (2001) J. Lipid Res. Vol 42, 1586-1593). It will therefore be appreciated that there is increasing evidence from epidemiological, clinical, and basic mechanistic studies to support the importance of HDL and apoA-1 in preventing or even reversing atherosclerosis. Therefore, finding drugs for increasing apoA-1 production by the liver and/or the small intestine, unique organs synthesising apoA-1, remains an attractive option for increasing HDL cholesterol in humans.

The present inventors have found that the orphan nuclear receptor liver receptor homolog-1 (hereinafter referred to as LRH1) directly regulates ApoA1 gene transcription. Thus LRH1 activators may provide a new therapy for the treatment of cardiovascular diseases.

### SUMMARY OF THE INVENTION

According to a first aspect the invention provides a method of treating or preventing a disease or condition caused by low plasma apoA-1 levels comprising administering a therapeutically effective amount of a liver receptor homolog 1 activator or a pharmaceutically acceptable salt, solvate or physiologically functional derivative thereof.

In a second aspect, the invention provides the use of a of a liver receptor homolog 1 activator or a pharmaceutically acceptable salt, solvate or physiologically functional derivative thereof in the preparation of a medicament for treating or preventing a disease or condition caused by low plasma apoA-1 levels.

### DESCRIPTION OF DRAWINGS

FIG. 1: Inhibition of LRH-1 Expression Results in Down-regulation of APOAl In HepG2 Cells

Panel A: HepG2 cells were transfected with increasing concentrations of a siRNA targeting LRH-1 or control siRNA (20 nM). 24 h post-transfection, LRH-1 mRNA levels were measured by quantitative RT-QPCR. Panel B: Western blot analysis of LRH-1 expression 24 h after transfecting HepG2 cells with various siRNAs (10 nM). Panel C: HepG2 cells were transfected with increasing concentrations of a siRNA targeting LRH-1. 48 hours post-transfection, APOAl mRNA levels were measured by RT-QPCR. APOB and SR-Bl gene expression was measured after 48 h transfection with the siRNA targeting LRH-1 (10 nM) or the non-silencing siRNA control (10 nM).

FIG. 2: Ectopic Expression of LRH-1 Induces APo Al gene expression in HepG2 Cells

Panel A: CV-1 cells were transfected with the human SHP promoter (100 ng) and increasing MOI of Ad-LRH-1 or Ad-null for 24 h in the presence or absence of a SHP expression vector (pSG5-SHP). Data are mean±SD of four individual transfections. Panel B: HepG2 cells were infected with Ad-LRH-1 or Ad-null (MOI=50). Total RNA was extracted and APOAl mRNA levels were measured by quantitative RT-QPCR. Data are mean±SD of three individual experiments.

FIG. 3: LRH-1 APOAl Expression at the Transcriptional Level

HepG2 (Panel A) or CV-1 cells (Panel B) were transfected with increasing amounts of an LRH-1 expression plasmid (0, 25, 50 or 100 ng) and the human APOAl promoter (100 ng). Panel C: Western blot analysis of 25 μg of total extracts derived from HepG2 or CV1 cells, was performed as described in ‘Materials and Methods’.

FIG. 4: Compound I Induces ADo Al Gene Expression in HepG2 Cells

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “physiologically functional derivative” refers to any pharmaceutically acceptable derivative of a LRH activator, for example, an ester or an amide, which upon administration to a mammal is capable of providing (directly or. indirectly) said LRH activator or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute of a LRH activator (or a salt or physiologically functional derivative thereof) and a solvent. Such solvents for the purpose of the invention may not interfere with the biological activity of the solute. Examples of suitable solvents include, but are not limited to, water, methanol, ethanol and acetic add. Preferably the solvent used is a pharmaceutically acceptable solvent. Examples of suitable pharmaceutically acceptable solvents include, without limitation, water, ethanol and acetic acid. Most preferably the solvent used is water.

The invention provides methods for treatment or preventing a disease or condition caused by low plasma apoA-1 levels, in particular atherosclerosis, dyslipidemia, peripheral vascular disease, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity and endotoxemia.

Preferably, the liver receptor homolog-1 (LRH1)activators are liver receptor homolog-1 (LRH1) agonists.

An LRH1 agonist is defined as a compound that increases coactivator peptide recruitment to the ligand binding domain by the method described herein.

Most preferably, LRH1 agonists are selective LRH1 agonists.

The following are further particular aspects of the present invention:
(a) A LRH1 activator for use as a therapeutic agent in the treatment of diseases or conditions, caused by low plasma apoA-1 levels.
(b) Pharmaceutical formulations comprising a LRH1 activator and at least one pharmaceutical carrier, wherein the LRH1 activator is present in an amount effective for use in the treatment or prevention of a disease or condition caused by low plasma apoA-1 levels.
(c) A method for identifying compounds that will be useful for the treatment of diseases or conditions caused by low plasma apoA-1 levels, comprising the step of determining whether a compound binds and/or activates LRH1.
(d) A method for treating diseases or conditions caused by low plasma apoA-1 levels comprising administration of a therapeutically effective amount of a compound (or salt, solvate or physiologically functional derivative thereof) that was identified as useful for such treatment by the above method (in other words, by a method comprising the step of determining whether a compound binds to and/or activates to LRH1).
(e) The use of a compound (or salt, solvate or physiologically functional derivative thereof) that was identified as useful for treating diseases or conditions caused by low plasma apoA-1 levels by the above method ( in other words, by a method comprising the step of determining whether a compound binds and/or activates LRH1), for the manufacture of a medicament for the treatment of a diseases or conditions caused by low plasma apoA-1 levels.

The term ‘treatment’ as used herein includes prophylaxis as well as alleviation of established diseases or conditions caused by low plasma apoA-1 levels. The LRH1 activators may be used as compounds or salts, solvates or physiologically functional derivatives thereof.

A suitable liver receptor homolog-1 (LRH1 )activator is shown below:

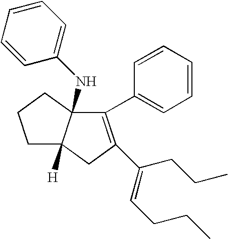

For details of the synthesis see David et al Synlett., 1994, (2), (6), 110-112. This compound is hereinafter referred to as Compound 1.

Those skilled in the art will recognize that stereocenters may exist in compounds which are LRH agonists. Accordingly, the present invention includes all possible stereoisomers and geometric isomers of such compounds in the methods and formulations of the present invention and includes not only racemic compounds but also the optically active isomers as well. When a compound is desired as a single enantiomer, it may be obtained either by resolution of the final product or by stereospecific synthesis from either isomerically pure starting material or any convenient intermediate. Resolution of the final product, an intermediate or a starting material may be effected by any suitable method known in the art. See, for example, Stereochemistry of Carbon Compounds by E. L. Eliel (Mcgraw Hill, 1962) and Tables of Resolving Agents by S. H. Wilen. Additionally, in situations where tautomers of the compounds are possible, the present invention is intended to include all tautomeric forms of the compounds.

It will also be appreciated by those skilled in the art that the LRH1 activator may also be utilized in the form of a pharmaceutically acceptable salt or solvate thereof. The physiologically acceptable salts include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References hereinafter to a LRH1 activator include both compounds and their pharmaceutically acceptable salts and solvates.

The LRH1 activators and their pharmaceutically acceptable derivatives are conveniently administered in the form of pharmaceutical compositions. Such compositions may conveniently be presented for use in conventional manner in admixture with one or more physiologically acceptable carriers or excipients. The carrier(s) must be “acceptable” in the sense of being compatible with the other ingredients of the formulation and not deleterious to the recipient thereof.

While it is possible that the LRH1 activators may be therapeutically administered as the raw chemical, it is preferable to present the active ingredient as a pharmaceutical formulation.

The formulations include those suitable for oral, parenteral (including subcutaneous e.g. by injection or by depot tablet, intradermal, intrathecal, intramuscular e.g. by depot and intravenous), rectal and topical (including dermal, buccal and sublingual) or in a form suitable for administration by inhalation of insufflation administration although the most suitable route may depend upon for example the condition and disorder of the recipient. The formulations may conveniently be presented in unit dosage form and may be prepared by any of the methods well known in the art of pharmacy. All methods include the step of bringing into association the compounds (“active ingredient”) with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a other conventional excipients such as binding agents, (for example, syrup, acacia, gelatin, sorbitol, tragacanth, mucilage of starch, polyvinylpyrrolidone) or hydroxymethyl cellulose or hydroxymethyl cellulose fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the LRH1 activators may be incorporated into oral liquid preparations such as aqueous or oily suspensions, solutions, emulsions, syrups or elixirs, for example. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid. Such preparations may also be formulated as suppositories, e.g., containing conventional suppository bases such as cocoa butter or other glycerides.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

For topical administration to the epidermis, the compounds may be formulated as creams, gels, ointments or lotions or as a transdermal patch.

The compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For intranasal administration the compounds of the invention may be used, for example as a liquid spray, as a powder or in the form of drops.

For administration by inhalation the compounds according to the invention are conveniently delivered in the form of an aerosol spray presentation from pressurised packs or a nebuliser, with the use of a suitable propellant, e.g. 1,1,1,2-trifluoroethane (HFA 134A) and 1,1,1,2,3,3,3,- heptapropane (HFA 227), carbon dioxide or other suitable gas. In the case of a pressurised aerosol the dosage until may be determined by providing a valve to deliver a metered amount. Capsules and cartridges of e.g. gelatin for use in an inhaler or insufflator may be formulated containing a powder mix of a compound of the invention and a suitable powder base such as lactose or starch.

In addition to the ingredients particularly mentioned above, the formulations may include other agents conventional in the art having regard to the type of formulation in question, for example those suitable for oral administration may include flavouring agents.

It will be appreciated by those skilled in the art that reference herein to treatment extends to prophylaxis as well as the treatment of established diseases or symptoms. Moreover, it will be appreciated that the amount of the LRH1 activator required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day. The formulations according to the invention may contain between 0.1-99% of the active ingredient, conveniently from 30-95% for tablets and capsules and 3-50% for liquid preparations.

The LRH1 activators for use in the instant invention may be used in combination with one or more other therapeutic agents for example, LXR ligands, FXR ligands, statins, and/or other lipid lowering drugs for example MTP inhibitors and LDLR upregulators. The compounds of the invention may also be used in combination with antidiabetic agents, e.g. metformin, sulfonylureas, or peroxisome proliferator activated receptor (PPAR) activators including PPAR gamma, PPAR delta, PPAR alpha PPAR alpha/gamma and PPAR pan agonists. The compounds may also be used in combination with antihypertensive agents such as angiotensin antagonists eg telmisartan, calcium channel antagonists eg lacidipine and ACE inhibitors eg enalapril . The invention thus provides in a further aspect the use of a combination comprising a LRH1 activator with a further therapeutic agent in the prevention or treatment of a disease or condition caused by low plasma apoA-1 levels.

When the LRH1 activators are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a LRH1 activator is used in combination with a second therapeutic agent active against the same disease, the dose of each compound may differ from that when the compound is used alone. Appropriate doses will be readily appreciated by those skilled in the art.

The following examples are set forth to illustrate the present invention. Accordingly, the following Example section is in no way intended to limit the scope of the invention contemplated herein.

### EXAMPLES

### Example 1

### LHR1 Upregulates APOA1

Cell Culture

HepG2 and CV1 cells (ATCC, Rockville, Md.) were maintained in Basic Eagle's medium (BME) supplemented with 2 mM glutamine, 1% non-essential amino acids and 10% (v/v) fetal calf serum (FCS) in an atmosphere of 5% CO₂ at 37° C.

Plasmids

The plasmids, pSG5-LRH-1, pSG5-SHP and pSG5-HNF4α have been previously described (Goodwin et al., (2000) Mol. Cell 6 517-526). The pSG5 plasmid, was purchased from Stratagene (La Jolla, Calif.). The human and mouse Apo Al promoter constructs (−1162+234) were obtained by PCR amplification using human and mouse genomic DNA (Clontech) as template. The resulting PCR products were inserted as a Bgl II/Hind III fragment into pGL3 basic vector (Promega) yielding hApo Al-Luc and mApo Al-Luc, respectively. The mutation of the LRH-1 binding site within human Apo Al promoter was obtained by site-directed mutagenesis (Stratagene, La Jolla, Calif.) using the following oligonudeotides: 5′-CAGAGCTGATCCTTTMCTCTTMG-3′ and 5′-CTTAAGAGTTAAAGGATCAGCTCTG-3′. All constructs were verified by DNA sequence analysis.

Transient Transfection Assays

HepG2 and CV1 cells, plated in 24-well plates at 50-60% confluence in BME supplemented with 10% FCS, were transiently transfected with reporter and receptor expression plasmids using Fugene 6 reagent (Roche Molecular Biochemical, Indianapolis, Ind.) as indicated in the Figure legends. The pSEAP2 expression plasmid (Clontech) was cotransfected to assess transfection efficiency. 48 hours post-transfection, cells were collected and assayed for luciferase and alkaline phosphatase activities. All experiments were repeated at least three times. Results are expressed as mean±SD.

RNA Interference

Twenty-one-nucleotide RNA oligonucleotides directed against human LRH-1 were obtained from Dharmacon, CO. Qiagen provided the non-silencing control siRNA. The siRNA sequence targeting LRH-1 (Genbank accession number AB019246) corresponds to the coding region (nucleotides 1199-1219) relative to the first nucleotide of the start codon. HepG2 cells (40% confluence) were transfected with siRNAs by using Translt-TKO reagent (Mirus, Madison, Wis.) following manufacturer's instructions. 24 hours post-transfecton, cells were refed with fresh medium for additional 24 hours.

RNA Analysis

Total cellular RNA was extracted using TRIZOL (Invitrogen). Real time quantitative PCR (RT-QPCR) assays were performed using an Applied Biosystems 7900 sequence detector. Total RNA (1 μg) was reverse transcribed with random hexamers using Taqman reverse-transcription reagents kit (Applied Biosystems) following the manufacturer's protocol. RNA expression levels were determined by Sybr green assays as described. 18SrRNA transcript was used as an internal control to normalize the variations for RNA amounts. Gene expression levels are expressed relative to 18S rRNA. Apo Al, Apo B, SR-Bl, SHP, LRH-1 and CYP7α mRNA levels were measured using the following oligonucleotides: For 18S: 5′-GGGAGCCTGAGAAACGGC-3′ and 5′-GGGTCGGGAGTGGGTATTT-3′; for APO Al: 5′-CTCGGCATTTCTGGCAGCAA-3′ and 5′-ACGTACACAGTGGCCAGGTCCTT-3′; for Apo B: 5′-TTCTGCCACATGCTTCCTCTT-3′ and 5′-GACCCGCCCCTTGTCAA-3′; for SR-Bl: 5′-TCCTCCGGGTCTTMAGGTGAT-3′ and 5′-GGCCTTTTGGTCCAGAATTTC-3′; for SHP: 5′-CGCCCTATCATTGGAGATGT-3′ and 5′-AGGAGCATTGGGTCACCTC-3′; for LRH-1: 5′-TGCAGGCTGMGAATACCTCT-3′ and 5′-GCATGCAACATTTCMTGAG-3′; for CYP7α:5′-CCCTTTGGATCGGGAGCTA-3′ and 5′-AGCTCCMTTCAAAATAAGAAAGCAT-3′.

Adenovirus Generation

The recombinant adenovirus (Ad-GFP and LRH-1) was obtained by homologous recombination in E. coli (Chartier et al., (1996) J. Virol 70 4805-4810) after insertion of the cDNAs into pAdCMV2. Viral stocks were created as previously described (Sardet et al., 1995). Viral titers were determined by a plaque assay on 293 cells and expressed as pfu/ml. Cells were infected, in most of the experiments, at a multiplicity of infection (MOI) of 50 viral particles per cell, by adding virus stocks directly to the HepG2 culture medium.

### Western Blot Analysis

Protein extracts were fractionated on 10% polyacrylamide gel under reducing conditions (sample buffer containing 10 mM dithiothreitol (DTT)), and transferred onto nitrocellulose membranes. LRH-1 protein was visualized by probing the membrane with a goat polyclonal LRH-1 antibody (SantaCruz, sc-6062). After incubation with a secondary peroxidase-conjugated antibody, signals were detected by chemiluminescence (Amersham, Buckinghamshire, UK).

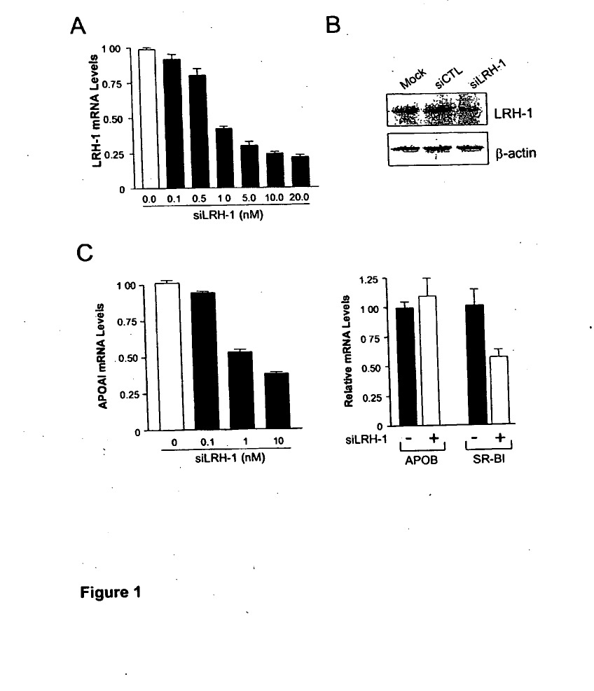

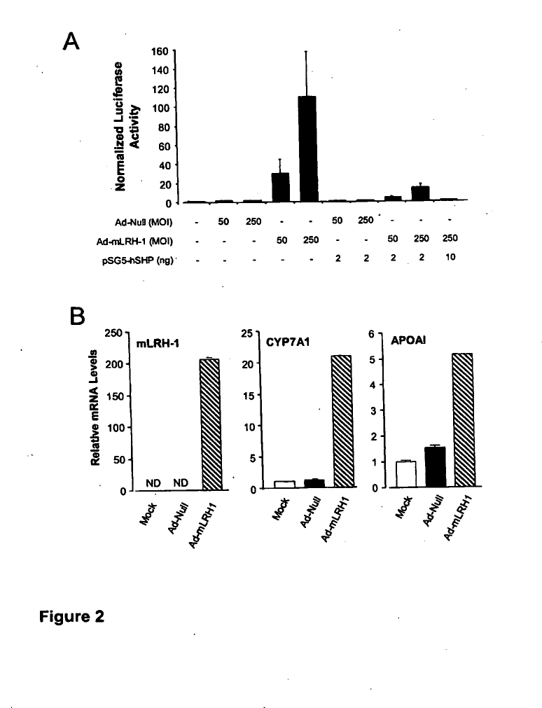

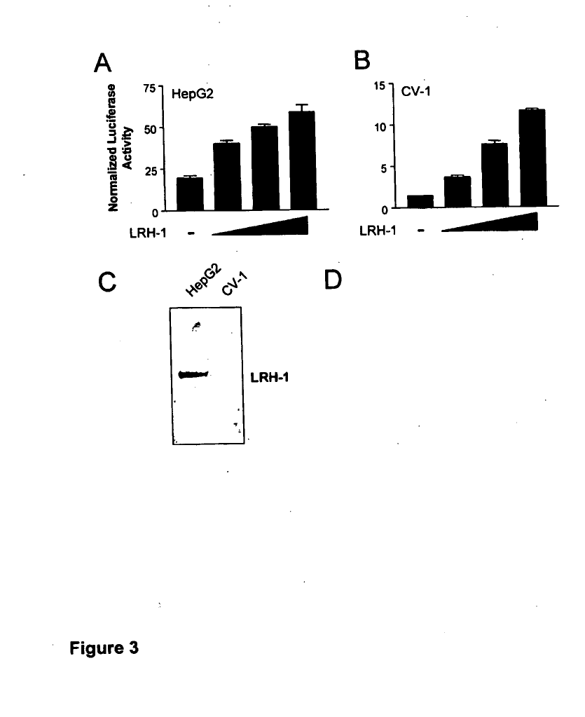

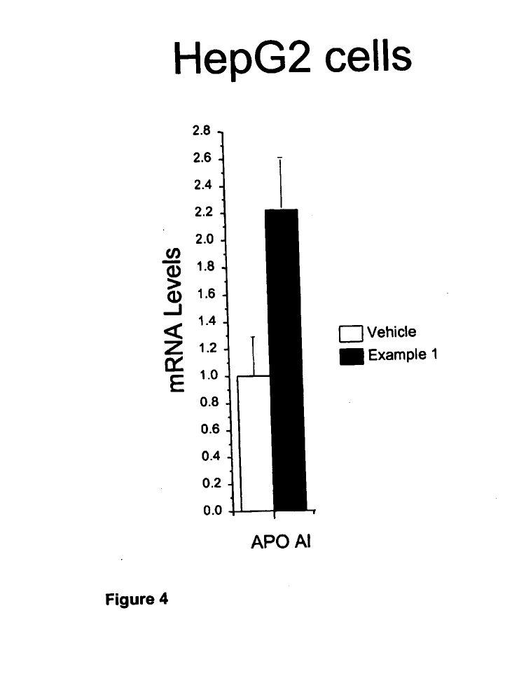

In order to identify novel LRH-1 target genes, gene-silencing experiments were performed in a liver-derived cell line, HepG2 cells, using RNA interference (RNAi) technology. Transfection of HepG2 cells with increasing concentrations of small interfering RNA (siRNA) targeting LRH-1 expression resulted in a dose-dependent inhibition of LRH-1 mRNA levels (FIG. 1A). Western immunoblot analysis revealed that this inhibition occurred also at the protein level and that the non-silencing control siRNA did not affect endogenous LRH-1 protein expression (FIG. 1B). As a control, β-actin protein levels were not modified after siRNA transfection (FIG. 1B). It was noted that APOAl gene expression was significantly decreased by a siRNA targeting LRH-1 expression (˜60% reduction). This inhibition appeared to be dose-dependent (FIG. 1C). In addition, SR-Bl, an established LRH-1 target gene (Schoonjans et al., 2002), was down-regulated in cells transfected with the siRNA targeting LRH-1 expression, whereas APOB gene expression was not affected demonstrating the specificity of this effect (FIG. 1C).

To further investigate a role for LRH-1 in APOAl gene regulation, an adenovirus encoding mouse LRH-1 (Ad-LRH-1) was generated by homologous recombination. To confirm the adenovirus construct was producing active LRH-1 protein, a kidney derived cell line known to express very low levels of LRH-1 (CV-1) was transfected with an LRH-1-responsive reporter gene construct, namely the human SHP promoter (bases −572 to +10), and infected with increasing amounts (multiplicity of infection; MOI) of virus. This resulted in a dose-dependent activation of the human SHP promoter (FIG. 2A). Furthermore, the LRH-1-dependent activation of this promoter was completely inhibited by over-expression of SHP. By contrast, a control virus expressing green fluorescent protein (Ad-Null) failed to activate this reporter gene construct. This result indicates that the adenovirus delivers a functional LRH-1 protein. Next, the influence of LRH-1 was tested on APOAl transcription in HepG2 cells. Ectopic expression of LRH-1 resulted in a robust (more than 5-fold) induction of APOAl mRNA in comparison to cells infected with the control virus (FIG. 2B). In parallel, expression of the CYP7Al gene was significantly (20-fold) increased by LRH-1 over-expression. These data suggests that LRH-1 regulates APOAl gene transcription in liver cells.

To determine the molecular mechanism by which LRH-1 regulates APOAl expression, a 1.4-kb fragment of the APOAl promoter was isolated and inserted upstream of a luciferase reporter gene. Transient transfection experiments performed in HepG2 cells revealed that LRH-1 dose-dependently induced APOAl promoter activity (FIG. 3A). A highly reproducible maximal induction of 2.5-fold was observed in this setting. By contrast, LRH-1 cotransfection in CV-1 cells led to a dramatic increase in of APOAl promoter activity (up to 11-fold). The more robust response observed in the CV-1 cells most likely reflects the lack of LRH-1 protein expression and lower basal activity of the APOAl-luciferase reporter observed in these cells (FIG. 3C). These results suggested that LRH-1 controls APOAl expression at the transcriptional level.

### Example 1

### LHR1 Activators Upregulate APOA1

Protein Expression

Human LRH-1 Ligand Binding Domain (LRH1 LBD) was expressed in E.coli strain BL21(DE3) as an amino-terminal polyhistidine tagged fusion protein. Expression was under the control of an IPTG inducible T7 promoter. DNA encoding this recombinant protein was subcloned into the pRSETa expression vector (Invitrogen). Sequence encoding the modified polyhistidine tag (MKKGHHHHHHG) was fused in frame to sequence encoding residues 248-501 of LRH-1. The coding sequence of Human LRH-1 LBD was derived from Genbank accession number U_{—}93553.

The resulting complete encoded sequence was as follows:

MKKGHHHHHHGSYMDSYQTSSPLKCEPDEPQVQAKIMAYLQQEQANRSKH
EKLSTFGLMCKMADQTLFSIVEWARSSIFFRELKVDDQMKLLQNCWSELL
ILDHIYRQVVHGKEGSIFLVTGQQVDYSIIASQAGATLNNLMSHAQELVA
KLRSLQFDQREFVCLKFLVLFSLDVKNLENFQLVEGVQEQVNAALLDYTM
CNYPQQTEKFGQLLLRLPEIRAISMQAEEYLYYKHLNGDVPYNNLLIEML
HAKRA

BL21 cells were transformed with the expression plasmid for LRH1 LBD and a single colony was used to inoculate 10 ml LB media containing 0.1 mg/ml Carbenicillin. The starter culture was grown overnight at 37 degrees C. and 1 ml aliquots were used to inoculate six flasks containing 1 liter of the same media. Cultures were grown with shaking for 24 hrs at 25 degrees C., harvested and frozen at −80 degrees C. until needed.

Purification of Receptor Ligand Binding Domain

25 g cell paste was resuspended in 400 mL TBS, pH 8.0 (25 mM Tris, 150 mM NaCl). Cells were lysed by passing 3 times through an APV Rannie MINI-lab homogenizer and cell debris was removed by centrifugation (30 minutes, 20,000 g, 4° C.). The cleared supernatant was filtered through coarse pre-filters, and TBS, pH 8.0, containing 500 mM imidazole was added to obtain a final imidazole concentration of 50 nM. This lysate was loaded onto a column (6×8 cm) packed with Sepharose [Ni++charged] Chelation resin (Pharmacia) and pre-equilibrated with TBS pH 8.0/50 mM imidazole. After washing to baseline absorbance with equilibration buffer, the column was washed with one column volume of TBS pH −8.0 containing 90 mM imidazole. LRH1 LBD was eluted with a gradient from 50 to 500 mM imidazole. Column peak fractions were pooled and dialyzed overnight against one liter TBS 8.0 containing 5 mM DTT and 0.5 mM EDTA. The dialyzed fractions were then diluted 5 fold with, 25 mM Tris pH 8.0, containing 0.5mM EDTA and 5mM DTT. The diluted protein sample was loaded onto a column (6×8 cm) packed with Poros HQ resin (anion exchange). After washing to baseline absorbance with the dilution buffer the protein was eluted with a gradient from 50-500 mM NaCl. Peak fractions were pooled and concentrated using Centri-prep 10K (Amicon) and subjected to size exclusion, using a column (3×90 cm) packed with Superdex-75 resin (Pharmacia) pre-equilibrated with TBS, pH 8.0, containing 0.5 mM EDTA and 5 mM DTT.

### Biotinylation of LRH-1 LBD

Purified LRH-1 LBD was desalted/buffer exchanged using PD-10 gel filtration columns into PBS [100 mM NaPhosphate, pH 7.2, 150 mM NaCl]. LRH-1 LBD was diluted to approximately 10 microM in PBS and five-fold molar excess of NHS-LC-Biotin (Pierce) was added in a minimal volume of PBS. This solution was incubated with gentle mixing for 60 minutes at ambient room temperature. The biotinylation modification reaction was stopped by the addition of 2000× molar excess of Tris-HCl, pH 8. The modified LRH-1 LBD was dialyzed against 4 buffer changes, each of at least 50 volumes, TBS containing 5 mM DTT, 2 mM EDTA and 2% sucrose, aliquoted, frozen on dry ice and stored at −80° C. The biotinylated LRH-1 LBD was subjected to mass spectrometric analysis to reveal the extent of modification by the biotinylation reagent. In general, approximately 95% of the protein had at least a single site of biotinylation; and the overall extent of biotinylation followed a normal distribution of multiple sites, ranging from one to five.

Assay

Reagents

Buffer: 5 E-2 M MOPS pH 7.5, 5 E-2 M NaF, 5E-5M CHAPS. 0.1 mg/ml Fraction 5 fatty acid free BSA

TIF2 Box3 peptide (B-QEPVSPKKKENALLRYLLDKDDTKD-CONH2)

In a polypropylene apparatus, a 4E-8 M (40 nM) solution of TIF2 Box3 peptide from a 1 E-4 M (100 uM) stock DMSO solution was added to a buffer to which 1E-8 M (10 nM) SA_europium (Wallac/perkin Elmer AD0063) had been added and the apparatus inverted gently to mix. The mixture was incubated for 15 minutes then treated with a 20 fold excess biotin, from a 1 E-2 M (10 mM) stock DMSO solution, and the apparatus invertes to mix. The resulting Europium-labelled TIF2 Box3 peptide preparation was incubated for 10 min and diluted 2 fold upon mixing with an equal volume of APC-labelled LRH-1 LBD (prepared below).

A 1E-8 M (10 nM) solution of biotinylated LRH-1 LBD was prepared in buffer to which 1 E-8 M (10 nM) SA-APC (Wallac/ Perkin Elmer CR130-100) had been added, and the mixture inverted gently to mix. This was incubated for 15 min then treated with a 20 fold excess biotin from a 1E-2 M (10 mM) stock DMSO solution, and inverted to mix. The resulting APC-labelled LRH-1 LBD preparation was incubated for 10min and diluted 2 fold upon mixing with an equal volume of Europium-labelled TIF2 Box3 peptide (prepared above).

The above solutions are gently mixed together to give a final preparation of 5E-9 M (5 nM) APC-labelled LRH1 LBD and 1E-8 M (20 nM) Europium-labelled TIF2 box3 peptide. After a 5 min incubation 25 ul of the solution was added to 384-well assay plates containing 1 ul of test compound (compound 1) in 100% DMSO. The plates were incubated for 1 hr then read on Wallac Victor2 V in Lance mode for EU/APC.

DATA ANALYSIS

To determine LRH1 agonist activity the APC (680 nM) counts were divided by the Europium (620 nM) counts. The resulting data were normalized using the following equation:

(unknown−ave background)/(ave basal−ave background)*100=% Basal inhibition/activation
unknown=test compound
Ave Background=excess TIF2 peptide
Ave Basal=no test compound

The nomalized data was fit to the equation:

Y=(Vmax*x)/(EC50+x)+Y2 EC50
Vmax=% activation or inhibition
Y2=basal

The results are shown below.

| | ave | | ave % | % basal |
|---|---|---|---|---|
| Compound 1 | pEC50 | stdev | basal | stdev |
| n of 44 | 6.59 | 0.214 | 277 | 91 |

Where EC50 is the half maximal concentration of the test compound that shows agonist activity, and ave % basal represented the maximum observed increase in agonist activity compared to the vehicle control.

### Example 3

### Compound I Induces APO Al Gene Expression in HepG2 Cells

HepG2 cells, cultured in 24-well plates, were treated for 48 hours with Compound I, 10 μm or vehicle (DMSO 0.1%) prior to harvest and determination of APOAl mRNA levels by RT-QPOCR as described in Example 1 above. The results are shown in FIG. 4.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims.

What is claimed is:

1. A method of treating diseases or conditions in a mammal, such as a human, disease or condition caused by low plasma apoA-1 levels which comprises the administration of a therapeutically effective amount of a LRH1 activator or salt, solvate or physiologically functional derivative thereof.

2. (canceled)

3. A method according to claim 1 wherein the LRH1 activator is a LRH1 agonist or salt, solvate or physiologically functional derivative thereof

4. A method according to claim 3 wherein the LRH1 agonist is a selective agonist or salt, solvate or physiologically functional derivative thereof

5. A method according to claim 1 where the LHR1 activator is a compound according to formula (I) or salt, solvate or physiologically functional derivative thereof

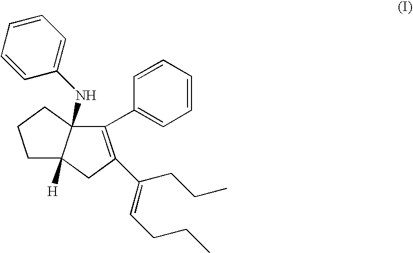

6. A method according to claim 1 wherein the disease or condition caused by low plasma apoA-1 levels is atherosclerosis, dyslipidemia, peripheral vascular disease, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity and endotoxemia.

7. A pharmaceutical formulation for use in the treatment of diseases or conditions caused by low plasma apoA-1 levels comprising a LRH1 activator or salt, solvate or physiologically functional derivative thereof together with at least one pharmaceutical carrier wherein the LRH1 activator is present in an amount effective for use in the treatment of diseases or conditions caused by low plasma apoA-1 levels.

8. A method for identifying compounds that will be useful in treating diseases or conditions caused by low plasma apoA-1 levels comprising the step of determining whether the compound interacts directly with LRH1, or the step of determining whether the compound activates LRH1.

9. A method for treating diseases or condition caused by low plasma apoA-1 levels comprising administration of compounds or salts, solvates or physiologically functional derivatives thereof identified using the screening method of claim 7.

10-11. (canceled)

12. A method of upregulating apoA-1 expression levels in vivo which comprises the administration of an effective amount of an LRH1 activator or salt, solvate or physiologically functional derivative thereof.

13. A method according to claim 12 where the LHR1 activator is a compound according to formula (I) or salt, solvate or physiologically functional derivative thereof [chemical structure]
